(19) United States
(12) Patent Application Publication
(10) Pub. No.: US 2005/0183976 A1
(43) Pub. Date: Aug. 25, 2005

(54) CRYOGENIC CANISTER ASSEMBLY

(76) Inventor: John G. Brothers, Clinton Township, MI (US)

Correspondence Address: GIFFORD, KRASS, GROH, SPRINKLE & CITKOWSKI, P.C, PO BOX 7021, TROY, MI, 48007-7021, US

(21) Appl. No.: 10/782,189

(22) Filed: Feb. 19, 2004

(51) Int. Cl. A61B 17/06

(52) U.S. Cl. 206/438

(57) ABSTRACT

A cryogenic canister assembly for biological specimens. The canister assembly includes a box having a lid and a base. The base has a bottom wall and sidewalls extending outwardly from the bottom wall around its outer periphery. Similarly, the lid includes a top wall and a sidewall extending outwardly from the top wall around the outer periphery of the top wall. Furthermore, the lid is dimensioned so that, when placed on the base, the lid sidewall overlies the base sidewall and forms a box having an interior chamber adapted to receive a plastic bag containing a biological specimen. A pair of U-shaped clips are insertable over opposed sides of the box to releasably lock the lid and base together. Both the lid and the base are constructed of cardboard.

### BACKGROUND OF THE INVENTION

I. Field of the Invention

The present invention relates to a cryogenic canister assembly.

II. Description of Related Art

Many medical and similar facilities include cryogenic freezers for containing biological specimens, such as blood, sperm and the like. These cryogenic freezers typically comprise a tank which is maintained at the appropriate cryogenic temperatures by liquid nitrogen or other means.

Typically, a plurality of biological specimens are contained in each cryogenic tank. Conventionally, a rack is insertable into the cryogenic tank and this rack is adapted to hold a plurality of cryogenic canister assemblies wherein each canister assembly holds one biological specimen.

These previously known canister assemblies typically comprised a lid having a base pivotally mounted to the lid and movable between an open and a closed position. In its closed position, the lid overlies the base and forms an interior chamber for the assembly. This interior chamber is adapted to hold a biological specimen, typically within a plastic bag.

In order to releasably lock the lid and base together, these previously known canister assemblies typically comprised a latch member pivotally mounted to the base and movable between a locked and an unlocked position. In its unlocked position, the latch allows the lid to pivot from its closed and to its open position to provide access to the biological specimen within the canister assembly or, alternatively, to place a biological specimen within the canister assembly. Conversely, with the latch in its locked position, the latch secures the lid to the base in its closed position thus retaining the biological specimen within the interior of the canister assembly.

These previously known canister assemblies have been typically constructed of aluminum. As such, the material cost for these previously known canister assemblies is very high, particularly since the canister assembly is oftentimes discarded after a single use. Additionally, the manufacturing cost for pivotally securing the lid to the base as well as pivotally securing the latch to the base further increases the overall cost of these previously known canister assemblies.

### SUMMARY OF THE PRESENT INVENTION

The present invention provides a cryogenic canister assembly which overcomes all of the above-mentioned disadvantages of the previously known devices.

In brief, the cryogenic canister assembly of the present invention comprises a box having a lid and a base. The base has a bottom wall and a sidewall extending outwardly from the bottom wall around the outer periphery of the bottom wall.

Similarly, the lid includes a top wall and a lid sidewall extending outwardly from the top wall around its outer periphery. Furthermore, the lid is dimensioned so that, when the lid is positioned over the base, the lid sidewall overlies the base sidewall and forms a box having an interior chamber. This interior chamber is adapted to hold a biological specimen, typically contained within a plastic bag.

A pair of U-shaped clips, each having a base leg and two spaced apart side legs, are insertable over opposed sides of the box. In doing so, one side leg of each clip overlies the top wall, the other side leg of each clip overlies the bottom wall, and the base leg overlies the lid sidewall thereby releasably securing the lid and base together.

Both the lid and the base are constructed of cardboard thus minimizing the cost of the cryogenic canister assembly without sacrifice as to its performance.

### BRIEF DESCRIPTION OF THE DRAWING

A better understanding of the present invention will be had upon reference to the following detailed description, when read in conjunction with the accompanying drawing, wherein like reference characters refer to like parts throughout the several views, and in which:

FIG. 1 is an elevational view illustrating the preferred embodiment of the present invention in its assembled form;

FIG. 2 is an exploded view illustrating a preferred embodiment of the present invention; and

FIG. 3 is a view taken substantially along line 3-3 in FIG. 1.

### DETAILED DESCRIPTION OF A PREFERRED EMBODIMENT OF THE PRESENT INVENTION

With reference to the drawing, a preferred embodiment of the cryogenic canister assembly 10 is shown and includes a box 12 having a lid 14 and a base 16. The base 16 includes a bottom wall 18 which is generally rectangular in shape and a sidewall 20 extending outwardly from the bottom wall 18 around its outer periphery.

Similarly, the lid 14 includes a top wall 22 which is also generally rectangular in shape and slightly larger than the bottom wall 18. A sidewall 24 extends outwardly from the top wall 22 about its outer periphery. Furthermore, as best shown in FIG. 3, with the lid 14 positioned on the base 16, the lid sidewall 24 overlies the base sidewall 20 and forms a chamber 26 between the lid 14 and base 16.

The chamber 26 is adapted to contain a biological specimen. Typically, the biological specimen, such as blood, is contained within a plastic bag 28 which is dimensioned to fit within the chamber 26 between the lid 14 and base 16.

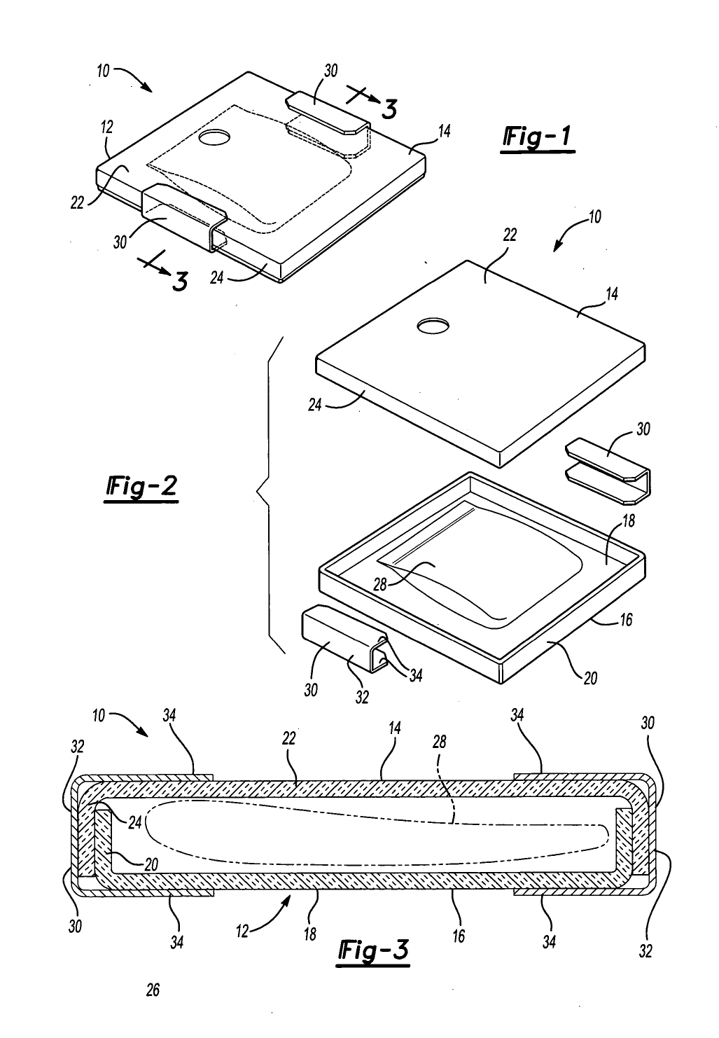

With the lid 14 positioned over the base 16 as illustrated in FIGS. 2 and 3, a pair of U-shaped clips 30 are provided for detachably securing the lid 14 and base 16 together. Each clip 30 includes a base leg 32 and two spaced apart side legs 34. Preferably, the clips 30 are constructed of aluminum and are dimensioned so that, with the clips 30 disposed over opposed sides of the box 12 as shown in FIGS. 1 and 3, one side leg 34 of each clip overlies the top wall 22, the other side leg of each clip 30 overlies the bottom wall 18, while the base leg 32 overlies the lid sidewall 24. Furthermore, the clips 30 are dimensioned so that, with the clips 30 positioned over opposed sides of the box 12, the side legs 34 of the clips 30 slightly compress the top wall 22 and bottom wall 18 towards each other thus frictionally securing the clips 30 to the box 12.

In order to minimize the cost of the canister assembly 10 without sacrificing performance, the lid 14 and base 16 are constructed of cardboard. In practice, however, the cardboard construction for the lid 14 and base 16 performs entirely satisfactorily even under cryogenic temperatures.

Having described my invention, however, many modifications thereto will become apparent to those skilled in the art to which it pertains without deviation from the spirit of the invention as defined by the scope of the appended claims.

1. A cryogenic canister assembly for biological specimens comprising:
a box having a lid and a base,
said base having a bottom wall and a base sidewall extending outwardly from said bottom wall around an outer periphery of said bottom wall,
said lid having a top wall and a lid sidewall extending outwardly from said top wall around an outer periphery of said top wall, said lid dimensioned so that said lid sidewall overlies said base sidewall and forms a box having an interior chamber adapted to contain a biological specimen,
a pair of U-shaped clips, each having a base leg and two spaced apart side legs, said clips insertable over opposed sides of said box so that one side leg of each clip overlies said top wall, the other leg of each clip overlies said bottom wall and said base leg overlies said lid sidewall,
wherein said base and said lid are of a cardboard construction.

2. The invention as defined in claim 1 wherein said clips are made of metal.

3. The invention as defined in claim 2 wherein said clips are made of aluminum.
